(19) United States
(12) Patent Application Publication
(10) Pub. No.: US 2002/0061298 A1
(43) Pub. Date: May 23, 2002

(54) Method for optimally delivering virus to a solid tumor mass

(76) Inventors: Matthew C. Coffey, Calgary (CA); Bradley G. Thompson, Calgary (CA)

Correspondence Address: Gerald F. Swiss, BURNS, DOANE, SWECKER & MATHIS, L.L.P., P.O. Box 1404, Alexandria, VA, 22313-1404, US

(21) Appl. No.: 09/987,687

(22) Filed: Nov. 15, 2001

Related U.S. Application Data
(60) Provisional application No. 60/252,221, filed on Nov. 20, 2000.

(51) Int. Cl. A61K 48/00; C12N 7/01

(52) U.S. Cl. 424/93.21; 435/235.1

(57) ABSTRACT

This invention relates to a method for optimally delivering virus to a solid tumor. Two approaches can be adopted to increase the number of tumor cells inside the solid tumor which are exposed to virus. The virus can be delivered to multiple sites inside the solid tumor, or the virus can be delivered to a single site in such a large volume that the delivered virus is capable of reaching more tumor cells in the solid tumor.

### RELATED APPLICATION

This application claims the benefit of U.S. Provisional Applications Serial No. 60/252,221, filed Nov. 20, 2000, the entire disclosure of which is hereby incorporated by reference.

### FIELD OF THE INVENTION

This invention relates to a method for optimally delivering virus to a solid tumor to increase the efficacy of oncolysis caused by the virus.

### REFERENCES

U.S. Pat. No. 6,110,461.

U.S. Pat. No. 6,136,307.

WO 94/18992, published Sep. 1, 1994.

WO 94/25627, published Nov. 10, 1994.

WO 99/08692, published Feb. 25, 1999.

Bar-Eli, N., et al., “preferential cytotoxic effect of Newcastle disease virus on lymphoma cells”, J. Cancer Res. Clin. Oncol. 122: 409-415 (1996).

Bischoff J R. et al., “An Adenovirus Mutant that Replicates Selectively in p53-Deficient Human Tumor”, Science 274(5286):373-6 (1996).

Blagoslelonny, M. V., et al., “in vitro Evaluation of a p53-Expressing Adenovirus as an Anti-Cancer Drug”, Int. J. Cancer 67(3):386-392 (1996).

Brooks et al., eds. “Jawetz, Melnick, & Adelberg's Medical Microbiology” (1998).

Chandron and Nibert, “Protease cleavage of reovirus capsid protein mu1 and mu1C is blocked by alkyl sulfate detergents, yielding a new type of infectious subvirion particle”, J. of Virology 72(1):467-75 (1998).

Chang et al., J. Virol. 69:6605-6608 (1995).

Chang et al., Virol. 194:537-547 (1993).

Chang et al., Proc. Natl. Acad. Sci. 89:4825-4829 (1992).

Coffey, M. C., et al., “Reovirus therapy of tumors with activated Ras pathway”, Science 282: 1332-1334 (1998).

Fueyo, J., et al., “A Mutant Oncolytic Adenovirus Targeting the Rb Pathway Produces Anti-Glioma Effect in Vivo”, Oncogene 19(1):2-12 (2000).

Fields, B. N. et al., Fundamental Virology, 3rd Edition, Lippincott-Raven (1996).

Haig, D. M. et al., Immunology 93:335-340 (1998).

He, B. et al, Proc. Natl. Acad. Sci. 94:843-848 (1997).

Heise, C. et al., “Replication-selective adenoviruses as oncolytic agents”, J. Clin. Invest. 105(7):847-51 (2000).

Kawagishi-Kobayashi, M. et al., Mol. Cell. Biol. 17:4146-4158 (1997).

Nemunaitis, J., Invest. New Drugs 17:375-386 (1999).

Reichard, K. W., et al., “Newcastle Disease Virus Selectively Kills Human Tumor Cells”, J. of Surgical Research 52:448-453 (1992).

Romano et al., Mol. Cell. Bio. 18(12):7304-7316 (1998).

Sharp et al., Virol. 250:302-315 (1998).

Stojdl, D. F., et al., “Exploiting Tumor-Specific Defects in the Interferon Pathway with a Previously Unknown Oncolytic Virus”, Nat. Med. 6(7):821-825 (2000).

Strong, J. E. and P. W. Lee, “The v-erbV oncogene confers enhanced cellular susceptibility to reovirus infection”, J. Virol. 70: 612-616 (1996).

Strong, J. E., et al., “Evidence that the Epidermal Growth Factor Receptor on Host Cells Confers Reovirus Infection Efficiency”, Virology 197(1): 405-411 (1993).

Strong, J. E., et al., “The molecular basis of viral oncolysis: usurpation of the Ras signaling pathway by reovirus”, EMBO J. 17: 3351-3362 (1998).

Yoon, S. S., et al., “An Oncolytic Herpes Simplex Virus Type I Selectively Destroys Diffuse Liver Metastases from Colon Carcinoma”, FASEB J. 14:301-311(2000).

Zorn, U. et al., “Induction of Cytokines and Cytotoxicity against Tumor Cells by Newcastle Disease Virus”, Cancer Biotherapy 9(3):22-235 (1994).

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Virus therapy, in particular reovirus therapy, is a new and selective cancer therapy (U.S. Pat. Nos. 6,110,461 and 6,136,307; Coffey et al., 1998). The receptor for the mammalian reovirus, sialic acid, is a ubiquitous molecule, therefore reovirus is capable of binding to a multitude of cells. However, most cells are not susceptible to reovirus infection and binding of reovirus to its cellular receptor results in no viral replication or virus particle production. This is probably the reason why reovirus is not known to be associated with any particular disease.

It was discovered recently that cells transformed with the ras oncogene become susceptible to reovirus infection, while their untransformed counterparts are not (Strong et al., 1998). For example, when reovirus-resistant NIH 3T3 cells were transformed with activated Ras or Sos, a protein which activates Ras, reovirus infection was enhanced. Similarly, mouse fibroblasts that are resistant to reovirus infection became susceptible after transfection with the EGF receptor gene or the v-erbB oncogene, both of which activate the ras pathway (Strong et al., 1993; Strong et al., 1996). Thus, reovirus can selectively infect and replicate in cells with an activated Ras pathway, which forms the basis of reovirus cancer therapy.

In order for a virus to replicate in the cancer cells, it is important that the virus is delivered efficiently to the tumor. However, traditional delivery methods have been problematic for virus therapy against solid tumors. Since tumor patients are often immune suppressed, the virus can cause diseases in them which do not normally happen to healthy animals. Further, solid tumors frequently are isolated from the general systemic circulation of the tumor patient. Therefore, it is not optimal to deliver virus systemically, which exposes the entire body of the patient to the virus and imposes the risk of undesired clinical conditions in the immune suppressed individuals, while the virus may not reach the solid tumor.

Another common method of delivery is intratumor injection. Theoretically, the virus injected into the tumor will replicate in the tumor cells to generate more virus particles, which in turn spread through the tumor and eventually replicate in every tumor cell. However, it was discovered that while a single injection of reovirus killed the tumor cells at the site of injection and caused local necrosis, other tumor cells may continue to proliferate outside of the site of injection and result in a net tumor growth. This result indicates that the spread of reovirus in solid tumors is not efficient enough. Therefore, a method for optimally delivering virus to a solid tumor is needed.

### SUMMARY OF THE INVENTION

This invention relates to a method for optimally delivering virus to a solid tumor. Two approaches can be adopted to increase the number of tumor cells inside the solid tumor which are exposed to the virus. The virus can be delivered to multiple sites inside the solid tumor, or the virus can be delivered to a single site in such a large volume that the delivered virus is capable of reaching more tumor cells in the solid tumor.

Accordingly, one aspect of the present invention is directed to a method for delivering a virus to a solid tumor to reduce growth of the tumor, comprising administering an effective amount of virus to a subject bearing the tumor by a base administration selected from the group consisting of:

(a) delivering a composition comprising the virus to multiple sites inside the solid tumor; and

(b) delivering directly into the tumor a composition comprising the virus, wherein the volume of the composition is between about 10% to about 100% of the volume of the tumor.

When the virus is delivered to multiple sites inside the tumor, it is preferable that at least 3 sites, more preferably at least 5 sites, are targeted. Most preferably, the number of delivery sites are determined by the volume of the tumor and the virus is delivered to one site per about 0.25 cubic centimeter of the tumor. This does not mean, however, that each delivery has to occur in a different 0.25 cubic centimeter. Nevertheless, the delivery sites are preferably as evenly distributed as possible.

When the virus is delivered in a large volume, the volume should be about 10% to about 100% of the volume of the tumor. It is preferable that the volume of the virus containing composition is at least 30% of the volume of the tumor. The virus is more preferably delivered in a volume which is at least 50% of the volume of the tumor.

The administrations at multiple sites and large volume are not mutually exclusive. It is contemplated that these two modes of administration may be combined. Moreover, in another aspect of this invention, the method can further comprise at least one more administration in addition to the base administration described above. The additional administration can be selected from the group consisting of:

(a) delivering a composition comprising the virus to multiple sites inside the solid tumor; and

(b) delivering directly into the tumor a composition comprising the virus, wherein the volume of the composition is between about 10% to about 100% of the volume of the tumor;

(c) delivering the virus by using a transdermal patch, a spray on the skin, or topical administration, wherein the tumor is a superficial tumor; and

(d) delivering the virus systemically.

The additional administration may be conducted concurrently with the base administration. Alternatively, the additional administration may be conducted at a different time from the base administration, for example on a day before or after the base administration.

It is contemplated that any virus, such as reovirus, which is capable of selectively replicating in tumor cells rather than normal cells is useful in the present invention. When reovirus is used, the reovirus is preferably a mammalian virus, more preferably a human reovirus, still more preferably a serotype 3 human reovirus, and most preferably a Dearing strain serotype 3 human reovirus. Other viruses which are useful in the present invention include, without being limited to, modified adenovirus, modified HSV, modified vaccinia virus, modified parapoxvirus orf virus, p53-expressing viruses, the ONYX-015 virus, the Delta24 virus, vesicular stomatitis virus, the herpes simplex virus 1 mutant which is defective in hrR3, Newcastle disease virus, encephalitis virus, herpes zoster virus, hepatitis virus, influenza virus, varicella virus, and measles virus.

### BRIEF DESCRIPTION OF THE DRAWINGS

FIG. 1 Various methods of viral delivery to a solid tumor.

(A) Single injection of virus to a solid lesion. Although a replication competent virus can spread from a central foci, tumor burden may still increase as a result of the high mitotic rate of the tumor cells on the periphery of the tumor mass.

(B) Multiple administrations (during a single interval or over an extended time period) increases the ability to reduce tumor burden by increasing the overall number of tumor cells infected. This is true even if the total number of infectious particles injected is the same as in a single injection.

(C) Increased number of infected cells can also be accomplished by increasing the delivery volume, even if the total number of infectious particles remains the same.

(D) Increased number of infected cells can also be accomplished via systemic delivery. Although vasculature that occurs during tumorigenesis does not allow for effective intratumoral delivery via this route, viruses in the circulation would contact and infect the rapidly growing cells in the outer portion of the tumor. Then viral spread would occur from the outside towards the inside.

FIG. 2 Local and systemic administration of reovirus

FIG. 2 shows the results of reovirus administration via various routes and injection schedules in immune-competent mice.

### DETAILED DESCRIPTION OF THE INVENTION

This invention relates to a method for optimally delivering virus, and particular reovirus, to a solid tumor. Traditionally, a therapeutic agent is injected as a single dose into a solid tumor. However, this method is not efficient in reovirus cancer therapy. Although reovirus is a replication competent virus and can spread from a central foci, tumor burden may still increase as a result of the high mitotic rate of the tumor cells on the periphery of the tumor mass. Therefore, this invention pertains to delivery of virus to more tumor cells, thereby increasing the infection efficiency, by using either of two approaches or the combination of both. The virus can be delivered to multiple sites inside the solid tumor, or the virus can be delivered in such a large volume that the delivered virus is capable of reaching more tumor cells in the solid tumor.

Prior to describing the invention in further detail, the terms used in this description are defined as follows unless otherwise indicated.

Definitions

A “tumor”, also known as a neoplasm, is a new growth comprising neoplastic cells. “Neoplastic cells”, also known as “cells with a proliferative disorder”, refer to cells which proliferate at an abnormally high rate. A neoplasm is an abnormal tissue growth, generally forming a distinct mass, that grows by cellular proliferation more rapidly than normal tissue growth. Neoplasms may show partial or total lack of structural organization and functional coordination with normal tissue. As used herein, a tumor is intended to encompass hematopoietic tumors as well as solid tumors.

A tumor may be benign (benign tumor) or malignant (malignant tumor or cancer). Malignant tumors can be broadly classified into three major types. Malignant tumors arising from epithelial structures are called carcinomas. Malignant tumors that originate from connective tissues such as muscle, cartilage, fat or bone are called sarcomas and malignant tumors affecting hematopoietic structures (structures pertaining to the formation of blood cells) including components of the immune system, are called leukemias and lymphomas. Other neoplasms include but are not limited to neurofibromatosis.

A method “to reduce growth of a tumor” means that upon application of this method, the tumor exhibits a lower weight or smaller size as compared to the same tumor not treated by this method. Preferably the growth of the tumor is reduced to such an extent that there is a net decrease in weight or size of the tumor.

An “effective amount” is an amount sufficient to result in the desired effect. The desired effect of the present invention is to reduce tumor growth. Therefore, an effective amount of virus is the total amount of virus administered which is sufficient to reduce tumor growth. Typically, the effective amount of virus is between 10³ and 10¹² plaque forming units (PFU). The effective amount in each case will vary with the species and size of the animal as well as the severity and nature of the tumor.

“Selectively killing tumor cells” or “selectively replicating in tumor cells” means that the virus preferentially kills or replicates in tumor cells rather than normal cells.

“Reovirus” refers to any virus classified in the reovirus genus, whether naturally occurring, modified or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reovirus have been recovered from many species. All three types share a common complement-fixing antigen.

The human reovirus consists of three serotypes: type 1 (strain Lang or T1L), type 2 (strain Jones, T2J) and type 3 (strain Dearing or strain Abney, T3D). The three serotypes are easily identifiable on the basis of neutralization and hemagglutinin-inhibition assays (see, for example, Fields, B. N. et al., 1996).

The reovirus may be naturally occurring or modified. The reovirus is “naturally-occurring” when it can be isolated from a source in nature and has not been intentionally modified by humans in the laboratory. For example, the reovirus can be from a “field source”, that is, from a human who has been infected with the reovirus.

The reovirus may be modified but still capable of lytically infecting a mammalian cell having an active ras pathway. The reovirus may be chemically or biochemically pretreated (e.g., by treatment with a protease, such as chymotrypsin or trypsin) prior to administration to the proliferating cells. Pretreatment with a protease removes the outer coat or capsid of the virus and may increase the infectivity of the virus. The reovirus may be coated in a liposome or micelle (Chandron and Nibert, 1998). For example, the virion may be treated with chymotrypsin in the presence of micelle forming concentrations of alkyl sulfate detergents to generate a new infectious subvirion particle.

The reovirus may be a recombinant (i.e., reassorted) reovirus from two or more types of reoviruses with differing pathogenic phenotypes such that it contains different antigenic determinants, thereby reducing or preventing an immune response by a mammal previously exposed to a reovirus subtype. Such recombinant virions can be generated by co-infection of mammalian cells with different subtypes of reovirus with the resulting resorting and incorporation of different subtype coat proteins into the resulting virion capsids.

“Adenovirus” is a double stranded DNA virus of about 3.6 kilobases. In humans, adenoviruses can replicate and cause disease in the eye and in the respiratory, gastrointestinal and urinary tracts. About one-third of the 47 known human serotypes are responsible for most cases of human adenovirus disease (Brooks et al., 1998). The adenovirus encodes several gene products that counter antiviral host defense mechanisms. The virus-associated RNA (VAI RNA or VA RNA_{I}) of the adenovirus are small, structured RNAs that accumulate in high concentrations in the cytoplasm at late time after adenovirus infection. These VAI RNA bind to the to the double stranded RNA (dsRNA) binding motifs of PKR and block the dsRNA-dependent activation of PKR by autophosphorylation. Thus, PKR is not able to function and the virus can replicate within the cell. The overproduction of virons eventually leads to cell death. The attenuated or modified adenovirus is unable to replicate in cells which do not have an activated Ras-pathway. However, attenuated or modified adenovirus can replicate in cells with an activated Raspathway.

The term “attenuated adenovirus” or “modified adenovirus” means that the gene product or products which prevent the activation of PKR are lacking, inhibited or mutated such that PKR activation is not blocked. Preferably, the VAI RNA's are not transcribed. Such attenuated or modified adenovirus would not be able to replicate in normal cells that do not have an activated Ras-pathway, however, it would be able to infect and replicate in cells having an activated Ras-pathway.

“Herpes simplex virus” (HSV) refers to herpes simplex virus-1 (HSV-1) or herpes simplex virus-2 (HSV-2). HSV gene _{γ1}34.5 encodes the gene product infected-cell protein 34.5 (ICP34.5) that can prevent the antiviral effects exerted by PKR. ICP34.5 has a unique mechanism of preventing PKR activity by interacting with protein phosphatase 1 and redirecting it activity to dephosphorylate eIF-2α (He et al., 1997). In cells infected with either wild-type or the genetically engineered virus from which the _{γ1}34.5 genes were deleted, eIF-2α is phosphorylated and protein synthesis is turned off in cells infected with _{γ1}34.5 minus virus. It would be expected that the _{γ1}34.5 minus virus would be replication competent in cells with an activated Ras pathway in which the activity of ICP34.5 would be redundant. HSV is unable to replicate in cells which do not have an activated Ras-pathway. Thus, HSV can replicate in cells which have an activated Ras-pathway.

The term “attenuated HSV” or “modified HSV” means that the gene product or products which prevent the activation of PKR are lacking, inhibited or mutated such that PKR activation is not blocked. Preferably, the HSV gene _{γ1}34.5 is not transcribed. Such attenuated or modified HSV would not be able to replicate in normal cells that do not have an activated Ras-pathway, however, it would be able to infect and replicate in cells having an activated Ras-pathway.

“Parapoxvirus orf virus” is a poxvirus. It is a virus that induces acute cutaneous lesions in different mammalian species, including humans. Parapoxvirus orf virus naturally infects sheep, goats and humans through broken or damaged skin, replicates in regenerating epidermal cells and induces pustular leasions that turn to scabs (Haig et al., 1998). The parapoxvirus orf virus encodes the gene OV20.0L that is involved in blocking PKR activity (Haig et al., 1998). The parapoxvirus orf virus is unable to replicate in cells which do not have an activated Ras-pathway. Thus, the parapoxvirus orf virus replicate in cells which have an activated Ras-pathway.

The term “attenuated parapoxvirus orf virus” or “modified parapoxvirus orf virus” means that the gene product or products which prevent the activation of PKR are lacking, inhibited or mutated such that PKR activation is not blocked. Preferably, the gene OV20.0L is not transcribed. Such attenuated or modified parapoxvirus orf virus would not be able to replicate in normal cells that do not have an activated Ras-pathway, however, it would be able to infect and replicate in cells having an activated Ras-pathway.

“Vaccinia virus” refers to the virus of the orthopoxvirus genus that infects humans and produces localized lesions (Brooks et al., 1998). Vaccinia virus encodes two genes that play a role in the down regulation of PKR activity through two entirely different mechanisms. E3L gene encodes two proteins of 20 and 25 kDa that are expressed early in infection and have dsRNA binding activity that can inhibit PKR activity. Deletion or disruption of the E3L gene creates permissive viral replication in cells having an activated Ras pathway. The K3L gene of vaccinia virus encodes pK3, a pseudosubstrate of PKR.

Deletion of residues which disrupt E3 function to inhibit the dsRNA binding. Additionally, since the amino terminal region of E3 protein interacts with the carboxy-terminal region domain of PKR, deletion or point mutation of this domain prevents anti-PKR function (Chang et al., 1992, 1993, 1995; Sharp et al., 1998; Romano et al., 1998). The K3L gene of vaccinia virus encodes pK3, a pseudosubstrate of PKR. There is a loss-of-function mutation within K3L. By either truncating or by placing point mutations within the C-terminal portion of K3L protein, homologous to residues 79 to 83 in eIF-2α abolish PKR inhibitory activity (Kawagishi-Kobayashi et al., 1997).

The term “attenuated vaccinia virus” or “modified vaccinia virus” means that the gene product or products which prevent the activation of PKR are lacking, inhibited or mutated such that PKR activation is not blocked. Preferably, the E3L gene and/or the K3L gene is not transcribed. Such attenuated or modified vaccinia virus would not be able to replicate in normal cells that do not have an activated Ras-pathway, however, it would be able to infect and replicate in cells having an activated Ras-pathway.

Method

We have developed methods for the efficient delivery of oncolytic viruses to solid tumors with the objective of increasing the number of tumor cells which are exposed to the virus. Oncolytic viruses are the viruses which are capable of selectively replicating in tumor cells but not normal cells, thereby causing the tumor cells to die. Accordingly, exposing more tumor cells to the virus can lead to more tumor cell death and higher efficacy of the virus therapy.

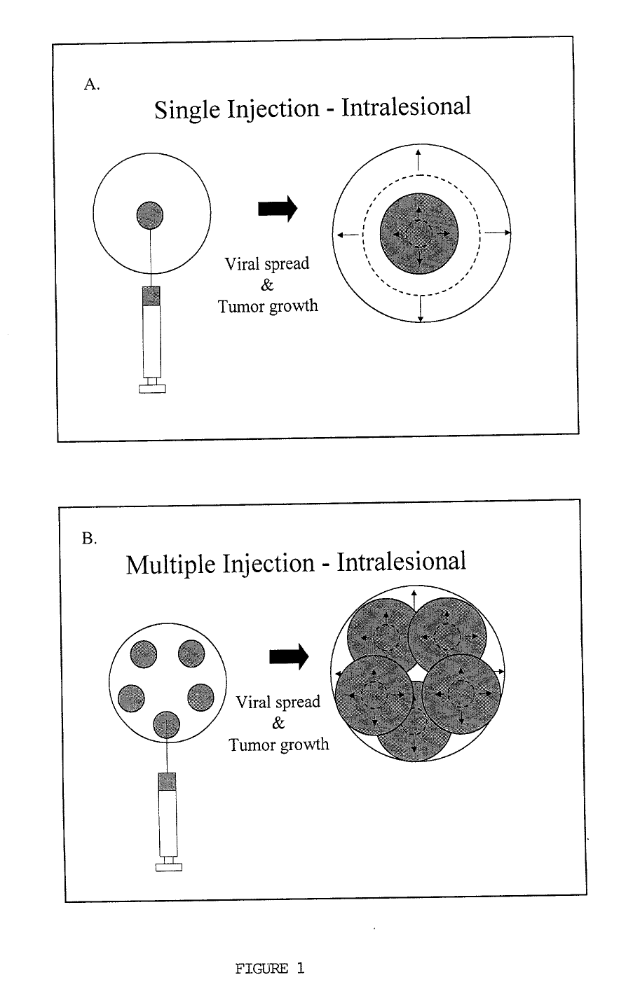

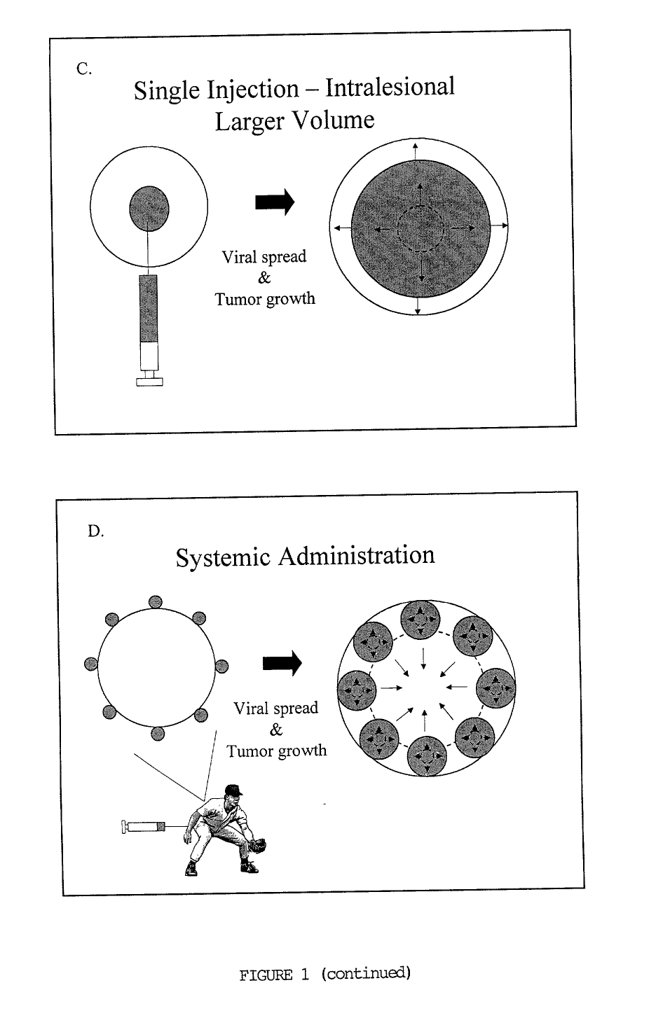

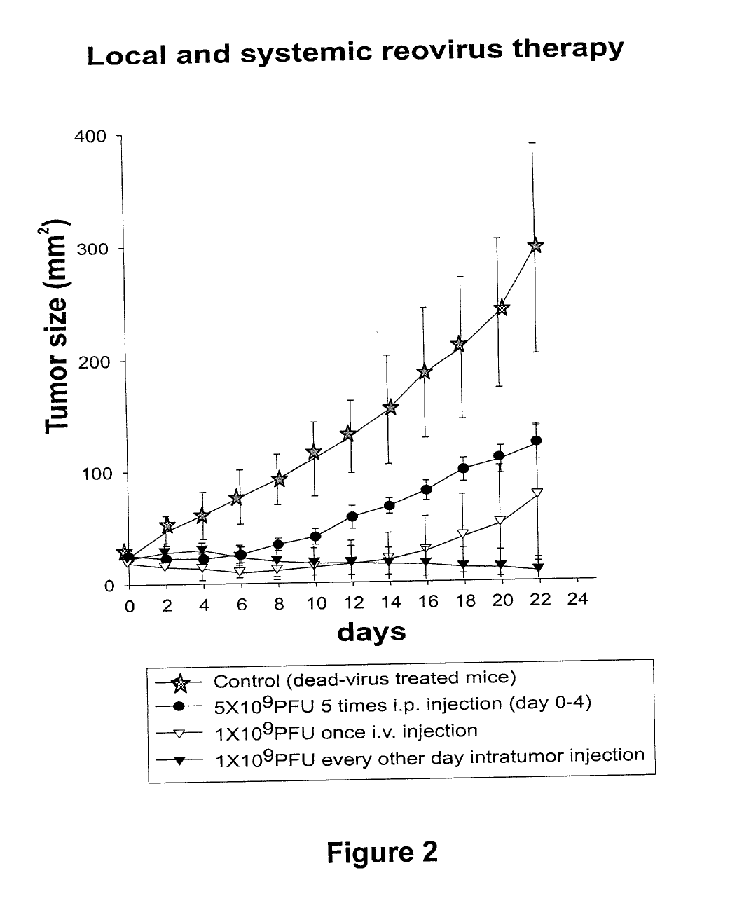

The traditional method of delivery a therapeutic agent to a solid tumor is to inject a single dose of the therapeutic agent into the tumor. As depicted in FIG. 1A, the cells around the injection site are exposed to the agent. The remaining cells, typically at the edge of the tumor, continue to grow. Since the cells on the outside of a solid tumor are the faster-growing cells, the ability of this method to reduce tumor growth is limited.

Increased number of virus-infected cells can be accomplished using systemic delivery. In this method, virus in the circulation may contact and infect the rapidly growing cells in the outer portion of the tumor. Once the cells on the outside are infected, the virus can spread to the inside of the tumor (FIG. 1D). However, as discussed above, this method unnecessarily exposes the whole body to the virus. Furthermore, if the tumor is blocked from the general circulation, the virus would not reach the tumor at all.

In the present invention, two methods are used to increase the efficiency of virus delivery to solid tumors. The virus can be injected at multiple sites within the tumor, particularly in the outer portion of the tumor. As shown in FIG. 1B, the virus can spread to a much larger portion of the tumor than a single injection, and tumor growth is restricted. Alternatively, the virus can be delivered to a single site in a large amount of fluid, which enables a wider spread of the virus (FIG. 1C). The optimal volume will need to be experimentally determined, but a minimum of 10% of the tumor volume is needed to achieve optimal results. It is preferable that the volume of the virus containing composition is at least 30% of the volume of the tumor. The virus is more preferably delivered in a volume which is at least 50% of the volume of the tumor. In order to determine the coverage area of the administration, a contrast agent or other indicator, such as a dye, can be added to the composition to facilitate detection of the administered composition.

The virus can be administered into the solid tumor in any suitable pharmaceutical excipient. To deliver at multiple sites, any device useful for multiple administration can be employed. For example, a direct injection can be repeated multiple times with a needle and syringe. Alternatively, a device with multiple injectors can be used to simultaneously inject at multiple sites.

Any virus which is capable of selectively replicating in tumor cells rather than normal cells is useful in the present invention. Examples of such a virus include reovirus, modified adenovirus, modified HSV, modified vaccinia virus, modified parapoxvirus orf virus, p53-expressing viruses, the ONYX-015 virus, the Delta24 virus, vesicular stomatitis virus, the herpes simplex virus 1 mutant which is defective in hrR3, Newcastle disease virus, encephalitis virus, herpes zoster virus, hepatitis virus, influenza virus, varicella virus, and measles virus. These oncolytic viruses are discussed below.

Adenovirus, HSV, vaccinia virus, and parapoxvirus orf virus are viruses which have developed a mechanism to overcome the double stranded RNA kinase (PKR). Normally, when virus enters a cell, PKR is activated and blocks protein synthesis, and the virus can not replicate in this cell. However, adenovirus makes a large amount of a small RNA, VA1 RNA. VA1 RNA has extensive secondary structures and binds to PKR in competition with the double stranded RNA (dsRNA) which normally activates PKR. Since it requires a minimum length of dsRNA to activate PKR, VA1 RNA does not activate PKR. Instead, it sequesters PKR by virtue of its large amount. Consequently, protein synthesis is not blocked and adenovirus can replicate in the cell.

Vaccinia virus encodes two gene products, K3L and E3L, which down-regulate PKR with different mechanisms. The K3L gene product has limited homology with the N-terminal region of eIF-2α, the natural substrate of PKR, and may act as a pseudosubstrate for PKR. The E3L gene product is a dsRNA-binding protein and apparently functions by sequestering activator dsRNAs.

Similarly, herpes simplex virus (HSV) gene _{γ1}34.5 encodes the gene product infected-cell protein 34.5 (ICP34.5) that can prevent the antiviral effects exerted by PKR. The parapoxvirus orf virus encodes the gene OV20.0L that is involved in blocking PKR activity. Thus, these viruses can successfully infect cells without being inhibited by PKR.

In the modified adenovirus, modified HSV, modified vaccinia virus, or modified parapoxvirus orf virus, the viral anti-PKR mechanism has been mutated or otherwise inactivated. Therefore, these modified viruses are not capable of replicating in normal cells which have normal PKR function. Ras-activated neoplastic cells, however, are not subject to protein synthesis inhibition by PKR, because ras inactivates PKR. These cells are therefore susceptible to infection by the modified adenovirus, modified HSV, modified vaccinia virus, or modified parapoxvirus orf virus.

The viruses can be modified or mutated according to the known structure-function relationship of the viral PKR inhibitors. For example, since the amino terminal region of E3 protein interacts with the carboxy-terminal region domain of PKR, deletion or point mutation of this domain prevents anti-PKR function (Chang et al., 1992, 1993, 1995; Sharp et al., 1998; Romano et al., 1998). The K3L gene of vaccinia virus encodes pK3, a pseudosubstrate of PKR. There is a loss-of-function mutation within K3L. By either truncating or by placing point mutations within the C-terminal portion of K3L protein, homologous to residues 79 to 83 in eIF-2α abolish PKR inhibitory activity (Kawagishi-Kobayashi et al., 1997).

Other oncolytic viruses include the viruses which selectively kill neoplastic cells by carrying a tumor suppressor gene. For example, p53 is a cellular tumor suppressor which inhibits uncontrolled proliferation of normal cells. However, approximate half of all tumors have a functionally impaired p53 and proliferate in an uncontrolled manner. Therefore, a virus which expresses the wild type p53 gene can selectively kill the neoplastic cells which become neoplastic due to inactivation of the p53 gene product. Such a virus has been constructed and shown to induce apoptosis in cancer cells that express mutant p53 (Blagosklonny et al., 1996).

A similar approach involves viral inhibitors of tumor suppressors. For example, certain adenovirus, SV40 and human papilloma virus include proteins which inactivate p53, thereby allowing their own replication (Nemunaitis 1999). For adenovirus serotype 5, this protein is a 55 Kd protein encoded by the E1B region. If the E1B region encoding this 55 kd protein is deleted, as in the ONYX-015 virus (Bischoff et al, 1996; Heise et al., 2000; WO 94/18992), the 55 kd p53 inhibitor is no longer present. As a result, when ONYX-015 enters a normal cell, p53 functions to suppress cell proliferation as well as viral replication, which relies on the cellular proliferative machinery. Therefore, ONYX-015 does not replicate in normal cells. On the other hand, in neoplastic cells with disrupted p53 function, ONYX-015 can replicate and eventually cause the cell to die. Accordingly, this virus can be used to selectively infect and kill p53-deficient neoplastic cells. A person of ordinary skill in the art can also mutate and disrupt the p53 inhibitor gene in adenovirus 5 or other viruses according to established techniques.

Another example is the Delta24 virus which is a mutant adenovirus carrying a 24 base pair deletion in the E1A region (Fueyo et al., 2000). This region is responsible for binding to the cellular tumor suppressor Rb and inhibiting Rb function, thereby allowing the cellular proliferative machinery, and hence virus replication, to proceed in an uncontrolled fashion. Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell.

Yet other oncolytic viruses include the interferon sensitive viruses. Vesicular stomatitis virus (VSV) selectively kills neoplastic cells in the presence of interferon. Interferons are circulating factors which bind to cell surface receptors which ultimately lead to both an antiviral response and an induction of growth inhibitory and/or apoptotic signals in the target cells. Although interferons can theoretically be used to inhibit proliferation of tumor cells, this attempt has not been very successful because of tumor-specific mutations of members of the interferon pathway.

However, by disrupting the interferon pathway to avoid growth inhibition exerted by interferon, tumor cells may simultaneously compromise their anti-viral response. Indeed, it has been shown that VSV, an enveloped, negative-sense RNA virus rapidly replicated in and killed a variety of human tumor cell lines in the presence of interferon, while normal human primary cell cultures were apparently protected by interferon. An intratumoral injection of VSV also reduced tumor burden of nude mice bearing subcutaneous human melanoma xenografts (Stojdl et al., 2000).

Other interferon-sensitive viruses (WO 99/18799), namely viruses which do not replicate in a normal cell in the presence of interferons, can be identified by growing a culture of normal cells, contacting the culture with the virus of interest in the presence of varying concentrations of interferons, then determining the percentage of cell killing after a period of incubation. Preferably, less than 20% normal cells is killed and more preferably, less than 10% is killed.

It is also possible to take advantage of the fact that some neoplastic cells express high levels of an enzyme and construct a virus which is dependent on this enzyme. For example, ribonucleotide reductase is abundant in liver metastases but scarce in normal liver. Therefore, a herpes simplex virus 1 (HSV-1) mutant which is defective in ribonucleotide reductase expression, hrR3, was shown to replicate in colon carcinoma cells but not normal liver cells (Yoon et al., 2000).

In addition to the viruses discussed above, a variety of other viruses have been associated with tumor killing, although the underlying mechanism is not always clear. Newcastle disease virus (NDV) replicates preferentially in malignant cells, and the most commonly used strain is 73-T (Reichard et al., 1992; Zorn et al, 1994; Bar-Eli et al, 1996). Clinical antitumor activities wherein NDV reduced tumor burden after intratumor inoculation were also observed in a variety of tumors, including cervical, colorectal, pancreas, gastric, melanoma and renal cancer (WO 94/25627; Nemunaitis, 1999).

Moreover, encephalitis virus was shown to have an oncolytic effect in a mouse sarcoma tumor, but attenuation may be required to reduce its infectivity in normal cells. Tumor regression have been described in tumor patients infected with herpes zoster, hepatitis virus, influenza, varicella, and measles virus (for a review, see Nemunaitis, 1999). According to the methods disclosed herein and techniques well known in the art, a skilled artisan can test the ability of these or other viruses to selectively kill neoplastic cells in order to decide which virus can be used to inhibit tumor growth using the methods of the present invention.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

°C.=degree Celsius

hr=hour

min=minute

μM=micromolar

mM=millimolar

M=molar

ml=milliliter

μl=microliter

mg=milligram

μg=microgram

PAGE=polyacrylamide gel electrophoresis

rpm=revolutions per minute

FBS=fetal bovine serum

DTT=dithiothrietol

SDS=sodium dodecyl sulfate

PBS=phosphate buffered saline

DMEM=Dulbecco's modified Eagle's medium

α-MEM=α-modified Eagle's medium

β-ME=β-mercaptoethanol

MOI=multiplicity of infection

PFU=plaque forming units

PKR=double-stranded RNA activated protein kinase

EGF=epidermal growth factor

PDGF=platelet derived growth factor

DMSO=dimethylsulfoxide

CPE=cytopathic effect

### Example 1

Local and Systemic Reovirus Administration

Immune-competent C3H mice were implanted with ras-transformed C3H-10T1/2 fibroblasts and allowed to develop tumors (Coffey et al., 1998). After tumor establishment, the mice were treated with Dearing strain reovirus via various routes and treatment schedules:

(A) intravascular injection of 1×10⁹ PFU on Day 0;

(B) intraperitoneal injection of 1×10⁹ PFU every day on Days 0-4 (5 injections); and

(C) intratumor injection of 1×10⁹ PFU every other day.

The control mice were treated with dead reovirus. Tumor sizes in all the animals were assessed every other day.

The results are shown in FIG. 2. Intraperitoneal injections, even when repeated 5 times, were not very effective in reducing tumor sizes. A one-time intravascular injection of reovirus was effective against the tumor for about 2 weeks, after which the tumors began to grow again. Multiple injections into the tumor, on the other hand, inhibited tumor growth effectively. Therefore, both local and systemic administrations of reovirus can be used in reovirus therapy.

### Example 2

Effect of Reovirus Administration to Multiple Sites of a Tumor

To study the effects of reovirus administration at multiple sites in a solid tumor, solid tumors are allowed to form in mice according to established methods in the art. For example, see Example 1 above, or Example 8 of U.S. Pat. No. 6,110,461.

Dearing strain reovirus is administered to the tumor-bearing mice according to the following courses of administration:

A. A single intratumor injection of 5×10⁹ PFU on Day 0;

B. 2 intratumor injections of 2.5×10⁹ PFU each (5×10⁹ PFU total) on Day 0;

C. 5 intratumor injections of 1×10⁹ PFU each (5×10⁹ PFU total) on Day 0; and

D. i.v. injection of 1×10⁹ PFU on Day 0.

In the control experiments, tumor-bearing mice are injected with dead reovirus. Tumor size is assessed every other day for each mouse. The results show that while every treatment course reduces tumor size as compared to the control, 5 intratumor injections are the most effective.

### Example 3

Reovirus Administration in Large Volumes

Tumors are allowed to form in mice as described above. The size of each tumor is estimated, and 5×10⁹ PFU of reovirus is prepared in a volume of vehicle equal to 10%, 20%, 30%, 50%, 75% and 100% of the tumor size, respectively, and injected into the tumor. One group of control mice receives dead reovirus formulated in the same manner. Another control group receives 5×10⁹ PFU of reovirus in 20 ul vehicle in the same manner Tumor size is then measured every other day.

The results indicate that larger volumes are more effective in reducing tumor sizes, particularly a volume 20-50% of the original tumor size.

We claim:

1. A method for delivering a virus to a solid tumor to reduce growth of the tumor, comprising administering an effective amount of virus to a subject bearing the tumor, wherein the virus is capable of selectively killing tumor cells, by a base administration selected from the group consisting of:
(a) delivering a composition comprising the virus to multiple sites inside the solid tumor; and
(b) delivering directly into the tumor a composition comprising the virus, wherein the volume of the composition is between about 10% to about 100% of the volume of the tumor.

2. The method of claim 1 wherein the virus is reovirus.

3. The method of claim 2 wherein the reovirus is a mammalian reovirus.

4. The method of claim 3 wherein the mammalian reovirus is a human reovirus.

5. The method of claim 4 wherein the human reovirus is a serotype 3 virus.

6. The method of claim 5 wherein the serotype 3 virus is a Dearing strain virus.

7. The method of claim 1 wherein the virus is selected from the group consisting of modified adenovirus, modified HSV, modified vaccinia virus, modified parapoxvirus orf virus, p53-expressing viruses, the ONYX-015 virus, the Delta24 virus, vesicular stomatitis virus, the herpes simplex virus 1 mutant which is defective in hrR3, Newcastle disease virus, encephalitis virus, herpes zoster virus, hepatitis virus, influenza virus, varicella virus, and measles virus.

8. The method of claim 1(a) wherein the virus is delivered to at least 3 sites inside the tumor mass.

9. The method of claim 1(a) wherein the virus is delivered to at least 5 sites inside the tumor mass.

10. The method of claim 1(a) wherein the virus is delivered to one site per about 0.25 cubic centimeter of the tumor.

11. The method of claim 1(b) wherein the volume of the composition is at least 30% of the volume of the tumor.

12. The method of claim 1(b) wherein the volume of the composition is at least 50% of the volume of the tumor.

13. The method of claim 1(a) wherein the total volume of the virus composition delivered is between about 10% to about 100% of the volume of the tumor.

14. The method of claim 1 further comprising at least one additional administration selected from the group consisting of:
(a) delivering a composition comprising the virus to multiple sites inside the solid tumor; and
(b) delivering directly into the tumor a composition comprising the virus, wherein the volume of the composition is between about 10% to about 100% of the volume of the tumor;
(c) delivering the virus by using a transdermal patch, a spray on the skin, or topical administration, wherein the tumor is a superficial tumor; and
(d) delivering the virus systemically.

15. The method of claim 14 wherein the at least one additional administration is conducted before or after the base administration.

16. The method of claim 14 wherein the at least one additional administration is concurrent with the base administration.

17. The method of claim 14 wherein the virus is reovirus.

18. The method of claim 17 wherein the reovirus is a mammalian reovirus.

19. The method of claim 18 wherein the mammalian reovirus is a human reovirus.

20. The method of claim 19 wherein the hum an reovirus is a serotype 3 virus.

21. The method of claim 20 wherein the serotype 3 virus is a Dearing strain virus.
